(19) United States
(12) Patent Application Publication
(10) Pub. No.: US 2021/0139977 A1
(43) Pub. Date: May 13, 2021

(54) Method for identifying RNA isoforms in transcriptome using Nanopore RNA reads

(71) Applicant: Hong Kong Baptist University, Hong Kong (HK)

(72) Inventors: Zhongying ZHAO, Hong Kong (HK); Runsheng LI, Hong Kong (HK)

(21) Appl. No.: 17/090,916

(22) Filed: Nov. 6, 2020

Related U.S. Application Data
(60) Provisional application No. 62/931,839, filed on Nov. 7, 2019.

(51) Int. Cl. C12Q 1/6874; C12Q 1/6827; C12Q 1/6837

(52) U.S. Cl. CPC C12Q 1/6874; C12Q 1/6837; C12Q 1/6827

(57) ABSTRACT

The present invention provide a method for identifying different isoform using long reads of RNA sequencing. The method includes assigning sequence tracks to a given gene locus based on long-read mapping against a reference genome wherein existing isoforms are also included as a sequence track, excluding long-read mappings that show few overlaps with existing exon or are in antisense to the given gene locus, clustering the sequence tracks based on a distance score Score 1, merging the sequence tracks with a cut-off based on the distance scores Score 1 between the sequence tracks, merging the sequence tracks if the distance score Score 1 is lower than 5%, clustering the retained sequence tracks based on a mutual distance score Score 2, merging the sequence track with a shorter length in the summed exons and correcting the resulting sequence tracks for intron/exon junctions to result in different isoforms.

### CROSS-REFERENCE TO RELATED APPLICATIONS

The present application claims priority from the U.S. provisional patent application Ser. No. 62/931,839 filed Nov. 7^{th}, 2019, and the disclosure of which is incorporated herein by reference in its entirety.

### FIELD OF THE INVENTION

The present invention relates to a method to study the transcriptome complexity in C. elegans or other species using reads from direct RNA sequencing or other sequencing method with ultra-long size.

### BACKGROUND OF THE INVENTION

Alternative splicing is hallmarks of eukaryotic transcriptomes. Over 90% of human genes show evidence of for alternative splicing. It plays a key role not only in cell fate specification, but also in development of higher organisms with sophisticated tissues, organs and developmental processes by expanding the complexity of transcriptome and thus of proteome. Aberrant splicing has been frequently linked to various diseases, including cancer, ageing, diabetes, abnormal nutritional response and neuronal disorders. In addition, the transcriptome is further subjected to various base modifications with different biological implications. Systematic detection of such modifications and understanding of their in vivo roles remains a significant challenge.

Identification of all type of transcripts produced by a genome is crucial for understanding the functional complexity of normal development and disease progression, but remains a challenging task even in an organism with a relatively small genome. For example, to facilitate annotation of the transcriptome of C. elegans or C. briggsae with a genome size of approximately 100 Mb, various datasets have been used, including ESTs, full-length cDNAs and RNA sequencing of (RNA-seq) of cDNA fragments using massively parallel sequencing. Short RNA-seq reads, typically shorter than 200 nt, have played a leading role in transcriptome annotation during the past decade. However, it is difficult to reconstruct and quantify alternative transcripts using short reads, which is further complicated by a requirement of an amplification step. Clearly, the ability to produce longer reads using native RNA molecule without amplification would minimize perturbation of transcript integrity, permitting capturing of full-length RNA molecules, which would be ideal for elucidating transcriptome complexity, including alternative splicing, alternative transcriptional start and ending, as well as the underlying biology. To this end, synthetic long-read RNA sequencing has been introduced, which relies on sub-pooling of full-length cDNAs followed by sequence amplification, fragmentation and assembly to produce a long read. The method has been shown to be able to recover many novel isoforms in humans and mice. However, the amplification and reverse transcription steps make it problematic for quantification and detection of native modifications. The current method of choice for profiling RNA methylation is RNA immunoprecipitation using modification-specific antibodies followed by reverse transcription and massively parallel sequencing. However, it provides poor resolution in terms of modification site. Third generation sequencing technology, for example, Pacific Biosciences (PacBio) RSII platform, is able to produce long read and detect DNA methylation based on polymerase kinetics during DNA synthesis, but a reverse transcription step is required for the sequencing RNA molecule. Therefore, direct sequencing of native RNA molecules is still not feasible.

Recently, Oxford Nanopore Technology (ONT) has developed a direct sequencing method for both DNA and RNA based on changes in ion current profile when a nucleotide passes through a nanopore. Due to its ultra-long read length, it has been adopted for many applications, including resolving repeats within human Y chromosome centromeres, improving the existing genome assembly, the rapid on-site sequencing of pathogens, and detecting 5-methylcytosine (5^{m}C) in the genomes of humans and yeast. Direct sequencing of single-molecule native RNA is expected to benefit transcript integrity by getting rid of the steps for reverse transcription and amplification. The DNA modifications detected with ONT are highly correlated with those from the bisulfate sequencing-based method. Because ONT relies on the change of profile in electric current to differentiate nucleotide bases, with appropriate positive and negative training data sets, the platform may be able to detect known or unknown modifications in native RNA molecules without any pre-treatment step.

Given a relatively high error rate of the long reads, using them to define transcriptome complexity is not trivial. Several methods have been developed to call transcript isoforms with a reference genome using long reads, including ToFu and SQANTI, which were designed for PacBio cDNA reads. These methods depend heavily on existing splicing junctions to classify the reads into representative isoforms, which may compromise the potential of the long read in defining novel splicing junction. Therefore, they demand precise junctions for each individual read track. To satisfy this requirement, the junctions must be pre-corrected for each read using existing junctions or massively parallel sequencing reads (referred to as short reads hereafter). A method for calling transcript isoforms without a reference genome has also been developed. However, the method suffers from a higher false positive rate, and is problematic in handling close paralogs, which are often associated with short reads. Importantly, with decreasing costs of third generation sequencing, it has become increasingly desirable to define the transcriptome complexity of an existing genome using long reads only. However, a method capable of meeting this challenge is still lacking.

RNA modifications are emerging as a significant player not only in regulation of rRNAs and tRNAs, but also in post-transcriptional regulation of mRNAs. More than 150 RNA modifications are known, but the true potential of only a few of these has recently been revealed at transcriptome scale, which is mainly due to rapid development in detection technology based on high-throughput sequencing. For example, transcriptome wide RNA modification is mainly achieved by coupling antibody immunoprecipitation or chemical treatments to massively parallel sequencing. However, these techniques suffer from low resolution or limited capacity for generalization. A more straightforward method for detection of transcriptome wide modification is necessary.

### SUMMARY OF THE INVENTION

The C. elegans genome is one of the best characterized metazoan genomes due to its homozygosity and lack of gaps. The 5′ end of most of its mRNAs carries a unique SL that is derived from independent loci, making it straightforward to evaluate the completeness of mRNA transcripts purified using oligo-d(T) magnetic beads. To examine the potential of ONT RNA sequencing in defining transcriptome complexity, direct sequencing of poly(A) tailed RNAs from different developmental stages of C. elegans is first performed in the present invention. Then a novel method for de novo identification of alternative splicing events by using the mapping tracks of full-length RNA transcripts is provided to identify 57,000 novel isoforms that are absent in the current annotation. Putative stage-specific expression of isoforms which was independent of the stage-specific expression of genes is also detected in the present invention. Finally, coding sequence-specific candidate RNA modification in all types of nucleotides is also shown in the present invention.

Accordingly, it is an objective of the present invention applying a direct RNA sequencing method with ultra-long reads using Oxford Nanopore Technologies to study the transcriptome complexity in C. elegans. Approximately six million reads using native poly(A) tailed mRNAs from three developmental stages, with average read lengths ranging from 900 to 1,100 nt are generated in the present invention. Around half of the reads represent full-length transcripts. To utilize the full-length transcripts in defining transcriptome complexity, a method is provided to classify the long reads as the same as existing transcripts or as a novel transcript using sequence mapping tracks rather than existing intron/exon structures, which identifies roughly 57,000 novel isoforms and recover at least 26,000 out of the 33,500 existing isoforms. Notably, the sets of gene with differential expression versus differential isoform usage over development are largely different, implying a fine-tuned regulation at isoform level during development. An unexpected increase in putative RNA modification in all bases in the coding region relative to the UTR is observed, suggesting their possible roles in translation. The ONT long reads and the method for read classification are expected to deliver new insights into RNA processing and modification and their underlying biology in the future.

In a first aspect of the present invention, there is provided a method for identifying different isoforms of RNA from a genome comprising: providing one or more nucleotide sequence reads from a nucleotide from an organism, wherein said one or more nucleotide sequence reads comprise at least one isoform; obtaining a reference sequence from an annotated database, wherein the reference sequence includes one or more gene loci having an exon and an intron; clustering one or more sequence tracks by a distance score Score 1 to obtain a first group of sequence tracks and a second group of sequence tracks; merging sequence tracks in the first group of sequence tracks if the distance score Score 1 is below a percentage of a first value; clustering sequence tracks in the second group of sequence tracks by a mutual distance score Score 2; and merging sequence tracks in the second group of sequence tracks if the mutual distance score Score 2 is below a percentage of a second value so as to generate one or more isoforms of the RNA with respect to one or more gene loci.

In a first embodiment of the first aspect of the present invention, there is provided a method wherein said clustering one or more sequence tracks by the distance score Score 1 comprises: constructing one or more sequence tracks by performing a read-mapping between the one or more nucleotide sequence reads and the one or more gene loci; excluding the sequence tracks having a first overlap to the exon below a certain amount of nucleotide, or excluding the sequence tracks having a second overlap to the antisense of the one or more gene loci; computing the distance score Score 1, wherein the distance score Score 1 is computed by

$$\frac{\begin{matrix}( {A}^{e x o n} ⋂ {B}^{e x o n} ) / ( {A}^{e x o n} ⋃ {B}^{e x o n} ) + \\ weight * ( {A}^{i n t r o n} ⋂ {B}^{i n t r o n} ) / ( {A}^{i n t r o n} ⋃ {B}^{i n t r o n} )\end{matrix}}{1 + weight} ,$$

wherein A^{exon} is an exon sequence of a first sequence track in the first group of sequence tracks, B^{exon} is an exon sequence of a second sequence track in the first group of sequence tracks, (A^{exon}∩B^{exon}) is the amount of shared exon sequence between A^{exon} and B^{exon}, (A^{exon}∪B^{exon}) is the amount of pooled exon sequence between A^{exon} and B^{exon}, A^{intron} is an intron sequence of the first sequence track in the first group of sequence tracks, B^{intron} is an intron sequence of the second sequence track in the first group of sequence tracks, (A^{intron}∩B^{intron}) is the amount of shared intron sequence between A^{intron} and B^{intron}, (A^{intron}∪B^{intron}) is the amount of pooled intron sequence between A^{intron} and B^{intron} and weight is a ratio of an average intron length to an average exon length of the reference sequence.

In a second embodiment of the first aspect of the present invention, there is provided a method wherein the certain amount of nucleotide according to the first embodiment is approximately from 10 to 100 nt.

In a third embodiment of the first aspect of the present invention, there is provided a method wherein if the distance score Score 1 according to the first embodiment is below the percentage of the first value, said merging sequence tracks in the first group of sequence tracks comprises: subtracting the length of the first sequence track and the length of the second sequence track in the first group of sequence tracks to obtain a third value; if the third value is over zero, merging the second sequence track with the first sequence track.

In a fourth embodiment of the first aspect of the present invention, there is provided a method wherein the ratio according to the first embodiment is approximately from 0.1 to 0.9.

In a fifth embodiment of the first aspect of the present invention, there is provided a method wherein the distance score Score 1 according to the third embodiment is below the percentage of approximately 5% of the first value.

In a sixth embodiment of the first aspect of the present invention, there is provided a method wherein said clustering sequence tracks in the second group of sequence tracks by the mutual distance score Score 2 comprises: computing the mutual distance score Score 2, wherein the mutual distance score Score 2 is computed by

$$\frac{\begin{matrix}( {A}^{e x o n} ⋂ {B}^{e x o n} ) / min ( {A}^{e x o n} , {B}^{e x o n} ) + weight * \\ ( {A}^{i n t r o n} ⋂ {B}^{i n t r o n} ) / min ( {A}^{i n t r o n} , {B}^{i n t r o n} )\end{matrix}}{1 + weight} ,$$

wherein A^{exon} is an exon sequence of a first sequence track in the second group of sequence tracks, B^{exon} is an exon sequence of a second sequence track in the second group of sequence tracks, (A^{exon}∩B^{exon}) is the amount of shared exon sequence between A^{exon} and B^{exon}, A^{intron} is an intron sequence of the first sequence track in the second group of sequence tracks, B^{intron} is an intron sequence of the second sequence track in the second group of sequence tracks, (A^{intron}∩B^{intron}) is the amount of shared intron sequence between A^{intron} and B^{intron}, and weight is a ratio of an average intron length to an average exon length of the reference sequence.

In a seventh embodiment of the first aspect of the present invention, there is provided a method wherein if the mutual distance score Score 2 according to the sixth embodiment is below the percentage of the second value, said merging sequence tracks in the second group of sequence tracks comprises: subtracting the first sequence track and the second sequence track in the second group of sequence tracks to obtain a fourth value; if the fourth value is over zero, merging the second sequence track with the first sequence track.

In an eighth embodiment of the first aspect of the present invention, there is provided a method wherein the ratio according to the sixth embodiment is approximately from 0.1 to 0.9.

In a ninth embodiment of the first aspect of the present invention, there is provided a method wherein the mutual distance score Score 2 according to the seventh embodiment is below the percentage of approximately 1% of the second value.

In a second aspect of the present invention, there is provided a method for identifying different RNA isoforms using long reads of RNA sequencing comprising assigning sequence tracks to a given gene locus based on long-read mapping against a reference genome wherein existing isoforms from the reference genome are also included as a sequence track, excluding long-read mappings that show few overlaps with existing exon from the existing isoforms or are in antisense to the given gene locus, clustering the sequence tracks based on a distance score Score 1 wherein the distance score Score 1

$$Score 1 = \frac{\begin{matrix}( {A}^{e x o n} ⋂ {B}^{e x o n} ) / ( {A}^{e x o n} ⋃ {B}^{e x o n} ) + \\ weight * ( {A}^{i n t r o n} ⋂ {B}^{i n t r o n} ) / ( {A}^{i n t r o n} ⋃ {B}^{i n t r o n} )\end{matrix}}{1 + weight}$$

wherein the amount of shared sequence in nucleotide between exons or introns from each sequence track is calculated as (A^{exon}∩B^{exon}) or (A^{intron}∩B^{intron}), and the total number of nucleotide (A^{exon}∪B^{exon}) or (A^{intron}∩B^{intron}) is calculated as pooled sequences in nucleotide between the same two exons or introns, and wherein the weight is based on the ratio of average intron length versus the exon length of the reference genome, with a default value of 0.5, merging the sequence tracks with a cut-off based on the distance score Score 1 between the sequence tracks, wherein the sequence track with a first length in summed exons of the isoforms other than the referenced isoforms is treated as a first subread and merged with the sequence track with a second length in the summed exons of said isoforms, wherein said second length is longer than the first length; merging the sequence tracks if the distance score Score 1 is lower than 5% and retaining one of the other isoforms with the biggest size of summed exons from each group along with the existing isoforms for subsequent isoform calling, wherein the remaining sequence tracks are assigned as a second subreads to be used for expression quantification and intron/exon boundary correction, clustering the retained sequence tracks based on their mutual distance score Score 2 wherein the mutual distance score

$$Score 2 = \frac{\begin{matrix}( {A}^{e x o n} ⋂ {B}^{e x o n} ) / min ( {A}^{e x o n} , {B}^{e x o n} ) + weight * \\ ( {A}^{i n t r o n} ⋂ {B}^{i n t r o n} ) / min ( {A}^{i n t r o n} , {B}^{i n t r o n} )\end{matrix}}{1 + weight} ,$$

merging the sequence track with a first length in the summed exons of the isoforms obtained after said clustering, being assigned as a third subreads, into a track with a second length in the summed exons of said isoforms if their mutual distance score Score 2 is lower than 1% so as to generate a one or more resulting isoforms for the given gene locus, wherein said second length is longer than the first length; correcting the resulting sequence tracks for intron/exon junctions to result in novel isoforms.

In a first embodiment of the second aspect of the present invention, there is provided a method wherein for said intron/exon boundary correction, the junctions from the second subreads are aligned against the junctions defined for the isoform by the longest full-length long-read.

In a second embodiment of the second aspect of the present invention, there is provided a method wherein a minor shift of no more than 5% change in distance score in exon-intron boundary caused by read errors are permitted to reduce over calling of different RNA isoforms.

Other aspects and advantages of the present invention will be apparent to those skilled in the art from a review of the ensuing description.

### BRIEF DESCRIPTION OF THE DRAWINGS

FIG. 1A shows fraction of full-length RNA reads (defined as reads with an SL signal or covering ≥95% length (indicated by dash line) of an annotated transcript) out of all RNA reads. Count distribution of read over its fraction of mapped length against existing transcript (WS260).

FIG. 1B shows proportion of reads over the fraction of their mapped lengths against those of the annotated transcripts derived from embryos (EMB), larvae (L1) and young adults (YA).

FIG. 1C shows distributions of read count over read length. The reads with and without splicing leader (SL) and annotation simulation curve was generated by replacing the actual length of long reads with the length of an annotated transcript to which it shows the highest similarity.

FIG. 1D shows distribution of full-length reads derived from mitochondrial genome. Top, read coverages of mitochondrial genes on the respiratory chain complex. Bottom: fraction of full-length reads for individual genes. Read count is shown proportional to the circle area.

FIG. 2A shows flowchart of new isoform calling by TrackCluster. Assignment of sequence tracks (indicated as grey bar with different numbers) to a given locus based on read mapping against the C. elegans genome. Existing isoforms are also included as individual tracks (black bar). Two reads that show few overlaps with any existing exons in or are antisense to a given locus are excluded from subsequent analysis.

FIG. 2B shows first round of clustering of tracks (including existing annotation) based on their distance scores (see Methods).

FIG. 2C shows read tracks (excluding existing transcripts) are merged if their distance scores satisfy our cutoff. Only the one with the biggest size of summed exons is retained (indicated with “✓”) from each group along with the existing one (indicated with “#”) for subsequent isoform calling. The remaining tracks (indicated with “x”) including existing transcripts are assigned as “subreads” and used only for expression quantification and boundary correction. Note, during track merging, a minor shift (indicated with “*”, within 5% change in “score 1” defined in Methods) in exon-intron boundary caused by read error is permitted to avoid over-calling of novel isoform.

FIG. 2D shows the retained tracks from the panel C are subjected to 2nd round of track clustering based on mutual distance scores (see “score 2” in Methods).

FIG. 2E shows the tracks (including existing transcripts) are merged if their distances satisfy our cutoff to avoid calling a novel isoform from a possible partially degraded read retained in panel C except for those starting with an SL.

FIG. 2F shows existing annotated (black) and newly called isoforms (grey) after junction correction (FIGS. 10A-10B). The retained track is called a novel isoform due to its distance score with any existing transcript satisfying our cut off.

FIGS. 2G-2I show schematic representation of each category of the newly identified isoform. Novel isoforms involving newly defined 5′ and/or 3′ end. “5′ and/or 3′ extra or missing” are/is defined as novel isoform with extra or missing exon at both or either end(s) of a novel isoform relative to an existing transcript. “UTR extensions” or “UTR truncations” is defined as novel isoform involving changes only in the UTR relative to an existing transcript: FIG. 2G shows the isoforms derived from alternative splicing and polyadenylation; FIG. 2H shows the isoforms derived from alternative promoters; FIG. 2I shows the isoforms derived from fully matched splicing junctions,

FIGS. 2J-2M show novel isoform involving the exon change within gene body: FIG. 2J shows the isoforms derived from exon inclusion or skipping; FIG. 2K shows the isoforms derived from intron retention; FIG. 2L shows the isoforms derived from alternative 5′ or 3′ splice sites; FIG. 2M shows the isoforms derived from gene fusion. Note that straightforward assignment of exon combination constitutes the main advantage of the long reads panel J.

FIG. 3A shows statistics of the newly called isoforms with long reads. Summary of the isoforms called using long reads. Left: bar plots of the number of existing isoforms that are recovered (defined as coverage of over 95% length of an existing transcript by at least a single long read, colored in black) or uncovered (the remaining isoforms, colored in grey). Right: bar plots of the number of novel isoforms called by TrackCluster. Breakdown of the novel isoforms is color-coded as in FIG. 2A-2F and is also shown at the bottom.

FIG. 3B shows abundance of the novel isoforms of various categories as defined in FIG. 2A-2F.

FIG. 3C shows length distribution of the novel isoforms of the various categories as defined in FIG. 2A-2F.

FIG. 3D shows count distribution of existing isoforms or the isoforms output by TrackCluster over their length.

FIG. 3E shows example of TrackCluster predicted isoforms. The accumulative long read coverage of unc-52 in all developmental stages was indicated. Three novel isoforms supported by the highest read coverage along with the existing isoforms (black) to which they bear the highest similarity. Genomic coordinates are shown on the top in kb.

FIG. 4A shows relationship between expression at gene level and isoform level during development. Correlations of expression at gene levels in three developmental stages determined by the long reads in this study and by the existing RNA-seq reads (WormBase release WS260).

FIG. 4B shows intersection of differentially expressed genes (DEG) and isoforms (DEI) between L1 and embryonic stages (L1_vs_EMB). Left: Venn diagram between DEG and DEI. Number of unique and shared genes are indicated. Right: Simulation of intersection between two sets of genes randomly chosen from the expressed genes either in L1 or embryos based on long reads. Number of genes sampled in each set is the same as that in DEG or DEI.

FIG. 4C shows venn diagrams showing the intersection of DEG (left) or DEI (right) among three stages.

FIG. 4D shows example of stage-specific abundance of TrackCluster predicted isoforms. Shaded in light blue is the coverage of long reads for gene efhd-1 over development. Existing (black) and TrackCluster predicted novel isoforms are shaded in grey. Novel isoforms are labelled as “1” to “5” as in FIGS. 2A-2F and existing isoforms are labeled as “a”, “b” or “c”). The existing isoform without any supporting long read is shown at the bottom. Note that the exon (indicated with arrowhead) with an elevated usage in young adult (55%) is highlighted in black box. The elevation is produced by stage-specific expression of the isoform “1”. The abundance of the isoforms in EMB, L1 and YA is indicated (5%, 9% and 55%, respectively).

FIG. 4E shows intersection of stage-specific expression between genes and isoforms. Venn diagram of the number of genes with stage-specific expression of isoforms.

FIG. 4F shows the upset plot showing the intersection between stage-specific expressed genes and isoforms.

FIG. 5A shows the long reads derived from embryos is significantly longer than those from L1 larvae or young adults. Boxplots showing the length of raw reads derived from embryos (EMB), L1 larvae (L1) and young adults (YA).

FIG. 5B shows boxplots showing the length of mapped reads from EMB, L1 and YA stages.

FIG. 5C shows boxplots showing the length of poly-A tails derived from EMB, L1 and YA stages.

FIG. 5D shows genes with a higher expression level (High) in embryo have an overall longer isoform. Boxplots showing the length of isoforms from three categories of genes, i.e., those that are expressed at a high (High), low (Low) or moderate (Mid) level in EMB than in both L1 and YA stages (see Methods). Note that the genes with elevated expression in the embryo tend to have a longer transcript. The results from existing WormBase annotation (WS260) and TrackCluster output.

FIG. 6A shows the percentage of putative modified bases along gene body in Embryos developmental stage. Putative base modifications are predicted with Tombo (version 1.4), and are differentially colored as indicated. The “de novo” model is used to identify all potential modifications on each base. The fraction of modification on each site indicates the normalized percentage of a putative modification out of all available reads on this site.

FIG. 6B shows Percentage of putative modified bases along gene body in L1 larvae developmental stage. Putative base modifications are predicted with Tombo (version 1.4), and are differentially colored as indicated. The “de novo” model is used to identify all potential modifications on each base. The fraction of modification on each site indicates the normalized percentage of a putative modification out of all available reads on this site.

FIG. 6C shows Percentage of putative modified bases along gene body in Young adults developmental stage. Putative base modifications are predicted with Tombo (version 1.4), and are differentially colored as indicated. The “de novo” model is used to identify all potential modifications on each base. The fraction of modification on each site indicates the normalized percentage of a putative modification out of all available reads on this site.

FIG. 7A shows comparisons of isoform characteristics between those derived from the long reads and those annotated in WormBase and those from Mangone et al, 2010 or those from meta-analysis of NGS RNA-seq data from Tourasse et al, 2017. Venn diagram shows the intersection of polyadenylation sites derived from at least five long reads and those from Mangone et al, 2010 or Tourasse et al, 2017.

FIG. 7B shows venn diagram showing the intersection of splicing junctions derived from TrackCluster results and those from WormBase annotation (WS260) or from Tourasse et al, 2017.

FIG. 7C shows Venn diagram showing the intersection of trans-splicing junctions derived from at least two Nanopore reads and those from Blumenthal et al, 2002 and Hwang et al, 2004 (downloaded from WormBase WS260) or from Tourasse et al, 2017.

FIG. 7D shows the fraction of full length read out of all reads for a given isoform over the length of the isoform.

FIG. 8 shows IGV track view of the reads mapped to the gene col-102. Notably, the 5′ end of the reads demonstrate decreasing length compared with a full-length annotated transcript shown on the bottom. Insertion and deletion are shown as “I” and a gap in the track, respectively.

FIG. 9 shows boxplots of the proportion of full-length reads out of all reads (x axis) against the isoforms of various lengths in nt (y axis). The median proportion decreases from approximately 95% to 85% for transcripts ranging from 200 to 3000 nt

FIG. 10A shows correction of intron-exon junction. Schematic diagram for the splicing junction correction. Shown here is a representative isoform 4 in FIGS. 2A-2F, which are associated with 3 subreads (3, 5, 9). The junctions from the subreads are aligned against the junctions defined in the isoform likely by a full-length long read. The shifted bps between the isoform and each subread are counted (shown at the bottom). A consensus junction was defined as the one with the highest frequency of support by all the subreads.

FIG. 10B shows distribution of junction shifting after correction. “0” indicates that the junction remains unchanged. Shifting to the 5′ and 3′ in bps relative to the isoform junction is indicated with minus and plus number, respectively.

FIG. 11A shows distribution of isoform number per gene. Bar plots showing count of genes with various number of isoform. Isoforms output by TrackCluster or WormBase are shown on the left and right, respectively.

FIG. 11B shows correlation between gene size and isoform. Boxplots show gene length in nt versus isoform number per gene.

FIG. 12A shows fusion isoforms. Venn diagram showing the intersection between the fusion isoforms output by TrackCluster and the operos annotated in WS260. Around 46% of the fusion isoforms are explained by operon.

FIG. 12B shows an example of two head-to-head fusion isoforms each from two adjacent transcripts that are known to be operonic ones.

FIG. 13A shows a missing exon sequence in the N2 genome is recovered judged by an insertion identified with long reads (0 kb-7 kb).

FIG. 13B shows a missing exon sequence in the N2 genome is recovered judged by an insertion identified with long reads (7 kb-14 kb).

FIG. 14A shows top 10 motifs for alternative PAS identified using long reads. Motifs identified from 351,437 PAS defined with at least two independent long reads (see Methods).

FIG. 14B shows top 10 motifs for alternative PAS identified using isoforms. Motifs identified from 44,700 isoforms output by TrackCluster.

FIG. 15 shows intersection of stage-specific expression between genes and isoforms. Upset plot shows the intersection between stage-specific expressed genes and isoforms. Six pairwise comparisons (shown on the left) are defined as individual group. The dot not associated with any line represents genes unique in this group. The dots linked with lines indicate groups used for intersection finding. Horizontal bars indicate gene number of each pairwise comparison. Vertical bars denote the numbers of intersection between groups.

FIG. 16 shows enriched GO terms for genes or isoforms with stage-specific expression. Shown are GO terms with a q value <0.1. Note that the sets of genes with differential expression show obvious overlap among various GO terms during development (comparison between the first two columns). However, sets of genes with differential isoform usage show little overlap among various GO terms during development (comparison between the first and the third or between second and fourth column). L1, L1 larvae; EMB, embryo; YA, young adult.

FIG. 17A shows fraction of accumulative modification of 5 mC along the gene body in three developmental stages: Embryo. Modifications of the nucleotide are predicted with Tombo (version 1.4) with “5^{m}C” model.

FIG. 17B shows fraction of accumulative modification of 5^{m}C along the gene body in three developmental stages: L1 larvae. Modifications of the nucleotide are predicted with Tombo (version 1.4) with “5^{m}C” model.

FIG. 17C shows fraction of accumulative modification of 5^{m}C along the gene body in three developmental stages: Young adult. Modifications of the nucleotide are predicted with Tombo (version 1.4) with “5^{m}C” model.

FIGS. 18A-18D show RNA modifications over development: FIG. 18A is Violin plots showing the distribution of weighted fraction of m5C (5-methylcytosine) type of modification in EMB, L1 nd YA stage. The black horizontal line indicates the 99% confidential interval of the distribution. The genes with weighted fraction higher than this level was defined as significantly modified genes; FIG. 18B is Venn diagram showing the intersection of the genes with significantly modified m5C between three different developmental stages; FIG. 18C is Violin plots showing the distribution of weighted fraction of modification of adenine “A” in EMB, L1 and YA stage. The black horizonal line indicates the 99% confidential interval of the distribution; FIG. 18D is Venn diagram showing the intersection of the genes with significantly modified “A” between three different developmental stages.

FIG. 19A shows retained introns which did not show obvious bias toward either end of gene body. Relative ratio of the isoforms involving retention of one (1), two (2) or three or more (3) introns among all “intron retention” isoforms.

FIG. 19B shows the average ratio of the retained introns alongside the relative position in gene body. The bin size for each isoform is 50 nt.

FIG. 19C shows venn diagram showing the intersection of gene numbers with intron retention supported by at least 2 reads in three developmental stages.

FIG. 20 shows boxplots showing the ratio of summed intron length over summed exon length derived from long reads in three developmental stages. Only the long reads with intron longer than 1000 nt was used.

FIG. 21A shows the length distribution of raw read length

FIG. 21B shows mapped read length.

FIG. 21C shows poly-A tail length of three developmental stages.

### DETAILED DESCRIPTION OF THE INVENTION

Throughout the present specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the present invention.

Furthermore, throughout the present specification and claims, unless the context requires otherwise, the word “include” or variations such as “includes” or “including”, will be understood to imply the inclusion of a stated integer or group of integers but not the exclusion of any other integer or group of integers.

In the methods of preparation described herein, the steps can be carried out in any order without departing from the principles of the invention, except when a temporal or operational sequence is explicitly recited. Recitation in a claim to the effect that first a step is performed, and then several other steps are subsequently performed, shall be taken to mean that the first step is performed before any of the other steps, but the other steps can be performed in any suitable sequence, unless a sequence is further recited within the other steps. For example, claim elements that recite “Step A, Step B, Step C, Step D, and Step E” shall be construed to mean step A is carried out first, step E is carried out last, and steps B, C, and D can be carried out in any sequence between steps A and E, and that the sequence still falls within the literal scope of the claimed process. A given step or sub-set of steps can also be repeated. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

Other definitions for selected terms used herein may be found within the detailed description of the present invention and apply throughout. Unless otherwise defined, all other technical terms used herein have the same meaning as commonly understood to one of ordinary skill in the art to which the invention belongs.

The present invention will be described in detail through the following embodiments with appending drawings. It should be understood that the specific embodiments are provided for an illustrative purpose only, and should not be interpreted in a limiting manner.

### Statistics of Read Length and Mappability

To evaluate the potential of direct RNA sequencing in identifying novel splicing isoforms, poly(A) tailed RNAs were first purified in the present invention. Most of these RNAs were expected to be mRNAs encoding proteins. Then direct RNA sequencing using portable MinION devices was performed and a total of approximately six million long reads from three developmental stages, i.e., embryo (EMB), L1 larva (L1) and young adult (YA) was generated. For each stage, at least 1.6 million reads with an average read length of 1118, 908 and 925 nt for EMB, L1 and YA were produced, respectively (Table 1). These reads are substantially longer than the short reads produced by massively parallel sequencing, which are typically less than 200 nt in length. The direct RNA sequencing reads were referred as long reads hereafter. It was expected that a subset of these reads represent full-length transcripts derived from the C. elegans genome.

TABLE 1
Read statistics

| Read characteristics | EMB | L1 | YA | Overall |
|---|---|---|---|---|
| Read number¹ | 1,638,628 | 1,829,380 | 2,440,814 | 5,908,822 |
| Average length | 1,118 | 908 | 925 | 974 |
| Median length | 916 | 718 | 729 | 765 |
| N50 length | 1,385 | 1,083 | 1,110 | 1,184 |
| Maximum read length | 20,913 | 22,897 | 20,145 | 22,897 |
| Average read quality | 11 | 11 | 11 | 11 |
| Average mapped | 1,072 | 855 | 914 | 940 |
| length² | | | | |
| Medium mapped length | 865 | 660 | 705 | 730 |
| Maximum mapped length | 15,710 | 15,577 | 16,152 | 16,152 |
| N50 of mapped reads | 1,347 | 1,069 | 1,107 | 1,169 |
| Average read accuracy | 84% | 83% | 86% | 85% |

¹The Reads number is the raw read number, including that of the ENO2 internal control.
²Based on Wormbase (WS260), containing a total of 33,501 protein-coding isoforms.

The reads were mapped against the C. elegans genome using minimap2 through “split-read” alignment, which implements “concave gap cost” for long insertions and deletions to accommodate intron skipping. Taking into account of mismatches and small insertions and deletions (indels) against the C. elegans genome, the overall read accuracy is roughly 85% (Table 1). Despite this relatively low read accuracy, the percentage of long reads that can be mapped back to the C. elegans genome, referred to as mappability hereafter, is 99.7%, indicating the high specificity of the long reads, consistent with previous mapping results using other types of long reads including PacBio cDNA reads. Despite a relatively low read quality score (average q score =11) of the long read compared with the short reads, this mappability is significantly higher than the short reads that are routinely used in RNA-seq, the mappability of which is around 80%. The substantially elevated mappability over an extended genomic interval provides an advantage in identification of novel splicing isoforms. Consistent with this, when the long reads were mapped against existing annotated spliced exons and UTRs and non-coding transcripts in the WormBase WS260, the overall mappability dropped to 76.7%. The nearly perfect mappability against the C. elegans genome contrasts sharply with a much lower mappability against its annotated transcripts, suggesting a possibility of novel transcripts that are currently missing in the WormBase, highlighting the value of the long reads in defining novel splicing isoforms.

To examine to what extent the long reads represent a full-length transcript, the long reads were classified into two categories, i.e., full-length and partial-length reads. Given that the mRNAs were purified using oligo-d(T) beads, most of them had intact 3′ ends. Therefore, the full-length reads were defined as those that span at least 95% of the length of their best hit of an existing transcript as described previously, or those that carry a splicing leader (SL) at 5′ end. The remaining reads were defined as partial-length reads (FIG. 1A). Over 65% of the SL sites (34,639 out of 52,846) identified using the long reads were also identified by meta-analysis of RNA-seq data or were currently annotated in WormBase (FIGS. 7A-7D). Approximately half of the long reads were defined as full-length ones with these criteria. YA showed a slightly higher percentage of full-length reads than L1 and EMB (FIG. 1B). Of the long reads, 23% carried SL (FIG. 1C). A previous analysis showed that approximately 80% of C. elegans protein-coding transcripts carried a SL at 5′ end. Judged by that benchmark, the percentage of SL-containing long reads in this study was lower than the expected 80% of the full-length reads, which would have corresponded to roughly 40% of the total long reads with the assumption that 3′ ends are intact. Two factors may have contributed to the underrepresentation of SL-containing reads. First, the mRNAs were purified from their 3′ end, which was expected to preferentially enrich reads that were intact at that end. Consistent with this, the 5′ but not 3′ end of the long reads seemed to undergo degradation (FIG. 8). Second, the technical issues associated with the ONT platform could have contributed to this phenomenon. The platform has been demonstrated to be problematic in resolving the very end of the last few nucleotides. This was further complicated with a relatively higher rate of read error by Nanopore platform than by massively parallel sequencing platform (Table 1).

To independently evaluate the capability of Nanopore long read in recovering full-length transcript, the percentage of full-length transcripts encoded by mitochondrial genes are calculated, which also carry a poly(A) tail but no intron in C. elegans, and it is expected few complications associated with splicing. Nearly 80% of the mitochondrial transcripts were full-length, although for ND5 the total was less than 70% (FIG. 1D), indicating that the long reads were able to recover majority of full-length transcripts that undergo no alternative splicing. The higher percentage of full-length transcripts from mitochondrial than from nuclear genes was probably due to their smaller size or lack of introns. Despite that the shorter genes tend to have a higher coverage of full-length reads, but the long reads were still able to cover 80% length of the transcripts for both mitochondrial and nuclear genes whose transcripts were up to 3,000 nt in length (FIG. 9). Only 5% existing nuclear genes were shown to produce transcript over 3,000 nt in length. The results highlight the value of these reads in resolving transcript complexity.

### A Pipeline for Reference Genome-Based Identification of Alternative Splicing Events

Current methods for identifying alternative splicing events using long reads mainly depend on predefined exon-intron junctions, leading to a high false positive rate in calling novel isoforms. For example, if an existing junction is inaccurately predicted, it will overwrite any isoforms defined by the long read mapping. In addition, existing methodologies for using long read to identify isoforms are not designed for quantifying expression level. Given the extremely high mappability of our long reads against the high-quality C. elegans genome, these full-length reads hold promise for de novo identification of intron-exon junctions, alternative promoter and polyadenylation site, as well as variation in UTR, which were collectively referred to as transcriptome complexity.

To take advantage of the long reads in defining transcriptome complexity, a new method, called TrackCluster, is provided to take full advantage of the mapping tracks of the full-length long reads to de novo construct a transcript isoform and determine its expression level. Using a customized classifier, TrackCluster either de novo identifies an isoform using a full-length transcript or groups the transcript with an existing isoform by their similarity score (FIGS. 2A-2M and FIGS. 10A-10B). To increase the confidence of calling of an isoform, it was demanded that the calling of an isoform must be simultaneously supported by at least five independent full-length long reads. Specifically, the mapping tracks of full-length reads were subjected to two rounds of clustering through calculating their intersection/overlapping scores (see Methods). The first round clustered all of the full-length reads with similar mapping tracks (exon/intron combinations) into distinct groups (FIGS. 2B-2C), whereas the second round merged the partial-length reads with an established group defined in the first round so as to quantify the expression level of the isoform (FIGS. 2D-2E). All of the transcript isoforms annotated in the WormBase WS260 were also included as a single track and clustered along with the long-read tracks. Therefore, TrackCluster not only outputs the isoforms that are consistent with the existing isoforms, but also holds promise to identify novel isoforms that could be missing from the existing annotations. As a result, TrackCluster outputs 12 categories of novel splicing isoforms relative to the existing isoforms to which they bear the highest similarity (FIGS. 2A-2M). Four of these categories involve alternative use of promoter or polyadenylation site, i.e., bearing extra or missing exon(s) at the 5′ or 3′ end. Another four categories involve UTR extensions or truncations at the 5′ or 3′ end, in which all of the newly identified intron-exon boundaries match with those of an existing isoform except the first or the last exon. To satisfy those latter four categories, the difference must be at least 5% of the summed length of all exons from the existing isoform. Two categories involve new combinations of exons within gene body, including extra or missing exon(s). One category involves intron retention, and the last category involves fusion of two separate isoforms from adjacent genes into a single isoform (FIGS. 2A-2F). Notably, for many of those with retained intron, they contained a premature codon. Part of them did have an intact open reading frame. However, the role of these isoforms were not known with the data only. Alternatively, TrackCluster is also able to de novo identify isoform independent of reference isoform, making it more useful for annotation of any newly sequenced genome.

To demonstrate the performance of “TrackCluster”, simulation datasets were generated for gene unc-52, for which 17 isoforms are currently annotated in the WormBase. For each isoform, 10-300 long reads were randomly generated. To mimic the characteristics of our actual reads, the reads with around 85% accuracy (3.9% mismatch, 6.1% deletion and 5% insertion) were generated, around 65% of which were expected to be in full length based on the statistics of our Nanopore reads. In addition, 23% of the full-length reads were marked with an SL signal. In 100 simulations, a FDR smaller than 5% in terms of novel isoform calling and a variation smaller than 10% in terms of isoform quantification were achieved.

### An Underestimation of Transcriptome Complexity in C. elegans

With approximately three million full-length long reads and approximately another three million partial-length long reads, 169,804 splicing junctions were totally identified. 150,591 (88.7%) out of those junctions were identical to those annotated in the WormBase (WS260). 4,537 (23.6%) of the remaining junctions (19,213) were also identified by meta-analysis of RNA-seq data (FIG. 7B). Consequently, approximately 25,000 (75%) out of existing 33,500 isoforms were recovered and a total of about 57,000 novel isoforms, which is approximately 1.5 to 3 folds with respect to the existing isoforms were identified and significantly expanded the complexity of existing isoforms annotated in the WormBase WS260 (FIG. 3A, FIGS. 11A-11B). Given our relatively low read coverage compared with the aggregated coverage of short RNA-seq reads used for exon annotation, it was not surprising approximately 7,000 existing isoforms were missed (FIG. 3A). The novel identifiable isoforms involved 11,921 genes. Given the summed exonic regions currently annotated in the WormBase are around 31.5M, about 4.99 M (15.8%) of these are missed by our long reads. Most of the novel isoforms were contributed by variations in the 5′ and/or 3′ end of the existing isoforms, i.e., alternative promoter or alternative polyadenylation (FIGS. 2G-2H). For example, it was frequently observed that an exon was missing at either end of a novel isoform relative to an existing transcript. It was also common that a UTR was expanded or truncated relative to an existing UTR. “5′ or 3′ extra or missing” was defined as a novel isoform with extra or missing exon at the 5′ or the 3′ end of a novel isoform relative to an existing transcript, respectively. Such variations in the 5′ and 3′ end were referred to as alternative promoter and alternative polyadenylation site, respectively. “UTR extensions or truncations” was defined as novel isoform involving extra or missing part only in the UTR relative to an existing transcript, respectively.

It is worth noting that nearly half of the members of the category of “fusion gene” (defined as fusion between two existing separate transcripts) belong to an operon (FIGS. 18A-18D), which serve as a nice validation of the identified isoform. These transcripts were likely captured before processing into two separate ones. Another half may contain some operonic transcripts that are currently unidentified. Part of the fusion transcripts could be derived from unidentified operonic transcripts. Notably, there were some reads that spanned two independent loci located even on different chromosomes. These reads were manually removed during calling of fusion isoform, resulting in a much smaller size of the category (FIGS. 2A-2C, 3A and 12A-12B). The category of “missing or extra exon” (defined as a isoform output by TrackCluster that shows a different exon combination relative to that of any existing transcript) within gene body contributes a relative small fraction of the novel isoforms, indicating that massively parallel sequencing-based RNA-seq analyses have been effective in recovering exons within gene body. However, the ability of straightforward assignment of exon combination highlights the main advantage of the long reads over the short reads. This is particularly true for genes with numerous exons and complicated splicing patterns. Notably, there are still 71,668 reads (around 1.2% of all our reads) that do not show obvious overlap with any existing isoforms. However, most of these reads are short with size smaller than 100, and these reads were not included in the analysis of isoform.

To examine whether different categories of novel isoforms demonstrated differential expression level, their accumulated expression level from the three developmental stages were plotted. The category involving UTR extensions demonstrated a higher expression level than the remaining categories (FIG. 3B), indicating that many isoforms differentiate themselves from others by changing their UTR. To evaluate whether the sizes of novel isoforms varied across categories, the length for all categories of novel isoforms were plotted. The results showed that the isoforms associated with exon change within gene body usually involved genes with a relatively large size (FIG. 3C). The one category that showed a significantly smaller size involved missing sequences in UTRs. Notably, the overall size of the novel isoforms identified by TrackCluster was comparable to that of the existing ones (FIG. 3D).

To help illustrate the use of long read coverage in identifying novel isoforms, three novel isoforms of unc-52 that were identified by TrackCluster with the highest coverage of long reads were depicted. Also shown were the three existing isoforms that have the highest level of similarity to the newly identified isoforms (FIG. 3E). One of the three novel isoforms was produced by skipping of multiple exons at its 3′ end relative to the existing isoform to which it has the highest level of similarity, i.e., the category of “3′ missing” (FIG. 2B). A careful examination of the accumulated coverage of the long reads showed that a sharp drop in one relevant exon compared with its neighbouring tracks. This exon was part of only one of the three novel isoforms (FIG. 3E). The remaining two isoforms skipped the exon relative to its closest reference isoform, i.e., they belonged to the category of “missing exon”.

In a few cases, the long reads were able to recover missing sequence within the C. elegans genome. For example, the long read derived from tsr-1 locus indicated absence of an exon along with its flanking sequences (FIGS. 13A-13B). Examination of the Illumina synthetic long reads produced previously showed that the exon along with its flanking intronic area was also missing in the current genome. By parsing the results from the read-to-genome alignments, it was able to obtain additional 730 loci that possibly carry a missing sequence longer than 50 nt. All these missing sequences were supported by at least five long reads. These loci involved 437 protein coding genes in total. Consistent with this, recent publication of the genome of an N2-derived strain VC2010 suggested that roughly 2% of the C. elegans encoded genes were affected by the deficiencies in the existing N2 reference genome. It remains a possibility that part of the missing sequences were produced by genetic variations unique to the strain used for sequencing.

The polyadenylation sites (PAS) from long reads form an independent resource for identification of its motif. The PAS site were determined as described (FIGS. 14A-14B). The canonical motif “AATAAA” consists of around a quarter of all PAS motifs. However, the PASs only show a moderate overlap with those of previous studies (FIG. 7A). This could be due to the difficulty of Nanopore reads in resolving the ion current signal of poly(A) sequence, which may lead to a slight shift of the boundary of the Poly(A) tail.

### The Sets of Genes with Differential Expression Versus Differential Isoform Usage are Largely Different

The capability to unambiguously assign isoforms using the long reads only permitted quantification of stage-specific expression not only at gene level, but also at isoform level. To evaluate whether stage-specific expression at gene level is contributed by differential expression of their isoforms, the isoforms from three developmental stages, i.e., EMB, L1 and YA, using TrackCluster were quantified. Despite a relatively high correlation of expression between gene levels measured with RNA-seq and the long reads (FIG. 4A), it was observed moderate overlap between the genes with stage-specific expression at gene and isoform levels (FIG. 4B, FIGS. 4E-4F and FIG. 15). In addition, most of the stage-specific genes were shared between the three stages, but fractions of the shared isoforms were much reduced at the isoform level (FIG. 4C). Gene Ontology analysis of stage-specific expression at gene and isoform levels also demonstrated little overlap between each other (FIG. 16), indicating that stage-specific expression at the gene and isoform level are largely uncorrelated from each other.

In one of the embodiment illustrating the power of the long reads in delineating stage-specific expression of isoform, the stage-specific read tracks along with the novel isoforms identified by the long reads as well as the existing isoforms for gene efhd-1 was plotted, which is an ortholog of human efhd-1 (EF-hand domain family member D1) (FIG. 4D). This gene encodes a calcium binding proteins, which are involved in variety of cellular processes such as mitosis, synaptic transmission, and cytoskeleton rearrangement. In addition, the expression of efhd-1 is increased significantly during neuronal differentiation. Diseases related to efhd-1 includes, for example, but not limited to Colorblindness, Partial, Protan Series and Red-Green Color Blindness. TrackCluster in the present invention identified a total of five isoforms supported by at least five long reads, while there were a total of four existing isoforms annotated in WormBase. Our isoform “1” showed the highest level of similarity to the existing isoform, efhd-16b. Approximately 55% of the long reads derived from YA were contributed by expression of the isoform “1”, whereas roughly 5% and 9% of the long reads from the EMB and L1 stages, respectively, were contributed by expression of that isoform. Therefore, the present invention provides a method for identifying different RNA isoforms including, for example, but not limited to fusion transcripts or types of abnormal transcript from a genome, which is useful as a diagnostic marker for cancer cells/tissues or genetic disease.

### Embryonic Transcripts are Longer than Postembryonic Transcripts

One unexpected observation was that the long reads derived from EMB were significantly longer in size than those of the remaining stages for both raw reads and mapped reads (Mann—Whitney U test, p<1e-15) (FIGS. 5A-5B, FIGS. 21A-21C). The poly(A) tails derived from EMB were also significantly longer than those of the remaining stages (Mann—Whitney U test, p<1e-15) (FIG. 5C). The sizes of both long reads and poly(A) tails were comparable between the L1 and YA stage. The size difference between the embryonic and postembryonic transcripts was not unique to the isoforms newly identified by the long reads (FIG. 5D). Existing transcripts annotated in the WormBase WS260 also showed a similar trend. The functional implications of the elevated size in embryonic transcripts remains to be determined.

### Elevated Putative RNA Modifications at Gene Coding Region

To explore the capability of direct RNA-seq to identify modifications in RNA molecules, all of the possible modified bases via the deviation of their ion current profile from that of known unmodified nucleotides was first identified by using Tombo. It worked by computing the possibility of a possible modification on each site for every read, and output the fraction of a possible modification on the site out of all input reads. The modification was detected by reproducible deviations of ion current profile of a base in question from that of an unmodified base without knowledge of exact chemical identity of the modification (FIGS. 17A-17C and 18A-18D). This was achieved with the assumption that the deviation in ion current profile of read error occurs randomly. The ratio of the modified bases were normalized against their relative position within the gene body and UTRs. Then the normalized ratio of each base along the gene body and UTRs were plotted. Previous study in C. elegans predominantly identified modified adenosines in the non-coding regions of DNA transposons. It was observed an apparent increase in the modification of all four types of base within the coding region relative to the UTR (FIGS. 6A-6C).

To investigate whether the modification in cytosine was contributed by 5^{m}C only, 5^{m}C was detected and quantified its ratio in RNA using a well-established method for 5^{m}C detection. The pattern of 5^{m}C is similar to that of total cytosine modifications but at a much smaller scale (FIGS. 6A-6C), suggesting that 5^{m}C contributed to a fraction of the observed ratio of modification in cytosine. The putative modifications in the bases A and U demonstrated opposite patterns from each other immediately before the start codon, whereas the modifications in all four bases except for U showed a sharp decrease immediately after the stop codon, suggesting that RNA modifications play an important role in protein translation and that U may have a unique role in termination of translation. In addition, the relative modification in U was higher in the YA stage than the remaining two stages, suggesting its stage-specific modification. It is worth noting that de novo detection of RNA modifications could be error prone, which should be treated with caution. It remains possible that the detected modification may be sequence context-dependent artifact without independent validation. Independent validation is required before working on the roles of the modified bases.

### Discussion

The capability of direct sequencing of full-length transcripts provides a key advantage over massively parallel sequencing-based RNA-seq analysis in several ways. First, direct sequencing of a full-length RNA transcript makes it straightforward to identify transcript isoform with numerous exons. Second, it enables profiling of developmental stage-specific or cell-specific expression of isoforms, which is problematic using RNA-seq, but may provide important insights into developmental processes. Finally, it holds promise to define RNA modifications de novo without extra treatment step, for example, by measuring the deviation in ion current profile from that of wild-type RNA. In the present invention, direct RNA sequencing of C. elegans poly-(A) tailed RNAs derived from three developmental stages was performed by using ONT technology, and it was provided a pipeline for identifying isoform variants based on read track, allowing straightforward characterization of transcriptome complexity in a stage-specific way.

### The Actual Number of Novel Isoforms could be even Higher

Approximately 75% out of the existing 33,500 isoforms were recovered, and identified another 57,000 novel isoforms with approximately 6 million long reads. It is likely that the actual number of novel isoforms may be substantially higher due to the following reasons. First, our sampling depth was not as high as those of analyses using RNA-seq both spatially and temporally. For example, three developmental stages were sampled in the present invention, whereas RNA-seq has been performed for tens of developmental stages in different cell or tissue types. Second, simultaneous support by at least five full-length reads were demanded to define a novel isoform. If this requirement was reduced to two full-length long reads, approximately 20,000 more candidate novel isoforms could be identified (data not shown). Third, the ratio of full-length reads may also be underestimated. This was because our definition of full-length was based on alignment against an existing transcripts. Many transcript isoforms may not exist in the WormBase that are currently annotated. For example, the full-length ratio of mitochondrial reads that carry no intron was 78%, whereas the ratio of nuclear mRNAs was only 49% (FIG. 1D). Although the proportion of full-length reads decreases slightly over transcript length (FIG. 9), this cannot account for the sharp decrease of full-length proportion between the mitochondrial and nuclear transcripts. It also suggests that a portion of the partial-length reads may be bona fide full-length ones but are present in the current WormBase. The main purpose of this study was not to capture as many isoforms as possible, but to demonstrate the capacity of the direct RNA sequencing technology in characterizing transcriptome complexity and in identifying novel RNA modifications. Future work should focus on systematic identification of alternative splicing events at different developmental stages and tissue/cell-types. This work also provides an entry point for biochemical and functional characterization of various RNA modifications observed in vivo.

### Intron-Retaining Isoforms seem to be Non-Functional

One category of novel isoform output by TrackCluster is “intron retention”, defined as a novel transcript that carries a well-established intron supported by various RNA-seq data (FIGS. 2A-2F). It was initially speculated that the retention of introns could be due to incomplete processing of pre-mRNAs, which were expected to retain multiple introns and to be enriched at the 3′ end, i.e., the end at which their processing ended. However, most (89.9%) of the isoforms with intron retention only retained one intron, and are shared between developmental stages, and location of the retained introns was enriched in the middle of gene body rather than at 3′ end (FIGS. 19A-19C), suggesting that these errors could be a product of incorrect rather than incomplete processing of pre-mRNA. Most of these intron-retaining isoforms carried a premature stop codon, suggesting that they were not functional in coding a protein.

### Complications Associated with Nanopore Direct RNA-Sequencing

It is possible that at least a fraction of the long reads could be artifacts. This is because that these reads contain sequences derived from different parts of a single chromosome or from different chromosomes. However, the chromosome assembly in the relevant regions seems to be intact, as judged by a lack of repetitive sequences or gaps in these regions. It was speculated that these reads could be chimeric ones created during adaptor ligation, i.e., two separate reads were ligated together. It was estimated that about 0.5% of the long reads were likely to be the results of such artefacts.

Despite the ultra-long read length offered by Nanopore direct RNA-seq, a few notable caveats might limit its application in the following respects. First, its sequence throughput is substantially lower than conventional RNA-seq, translating into a much higher sequencing cost per nucleotide. Currently, only one to two GB of data of RNA sequences can be generated per flow cell, whereas over 10 GB of data of DNA sequences can be produced using the same flow cell. This low throughput significantly inhibits its application in research areas that are heavily dependent on gene expression profiling, which demand an especially high coverage for these low-abundance transcripts. Second, the relatively high error rate in read sequences is problematic during alignment in some cases. A customized alignment algorithm must be used to accommodate these errors. Third, Nanopore direct RNA-seq is known to be deficient in calling the very last bases that it sequences. This could have contributed to our lower than expected percentage of calling of SL-containing transcripts. Given that Nanopore direct RNA-seq produces sequence from 3′ to the 5′ end, it was speculated that the underrepresentation of SL signals was partially due to incomplete sequence at the 5′ end of the long reads, which inhibited reliable calling of SL signals, typically only 22 nt in length. Fourth, methodology for detecting RNA base is in its infancy and under active development. A more robust method is needed to reliably detect the RNA base modification and its chemical identity. Any putative RNA base modifications reported in this study could be artifact resulting from various noises. Functional characterization of these modifications is not warranted until they are independently validated. Finally, Nanopore direct RNA-seq demands a large amount of starting RNAs in a magnitude of ˜100 microgram. This limits its use in single-cell analysis. Future development should focus on adaptation of Nanopore direct RNA-seq to small amount of starting materials, which would maximize its potential in identifying novel isoforms.

Taken together, with the newly provided classifying method in the present invention, the long reads generated by ONT greatly facilitate the unambiguous resolution of alternative splicing events. The reads also hold great potential in de novo identification of RNA modifications, which is expected to catalyse the functional characterization of the new isoforms and modifications. Given the evidences of conserved splicing events between nematode and mammals, part of the splicing events were expected to be conserved across species.

### METHODS

### Purification and Sequencing of mRNAs with MinION

Animals were as described and synchronized as embryo, L1 and young adult following a previous study. Briefly, C. elegans (N2) was cultured on NGM plates with OP50 E. coli at 20° C. Gravid adult worms were treated with bleach to isolate embryos. The embryos were incubated in M9 buffer without food at room temperature for 12 h to hatch and arrest at the L1 stage for harvesting. Part of the starved, synchronized L1 larvae were fed with OP50 and cultivated at 20° C. till adulthood to be harvested for RNA preparation. Animals of different stages, i.e., embryo, L1 larva and young adult, were collected and total RNAs were extracted using TRIzol (Invitrogen) following the manufacturer's instructions. Approximately 100 μg total RNAs were extracted for each sample. Around 900 ng of poly(A) tailed mRNAs was purified using Dynabeads™ mRNA Purification Kit (Invitrogen) based on the user's manual for each library preparation. Nanopore sequencing libraries were constructed using Direct RNA sequencing kit (cat #SQK-RNA001). The libraries were loaded onto Nanopore R9.4.1 flow cell (cat #FLO-MIN106) and sequenced on MinION acquired from Oxford Nanopore Technologies. The software used for sequencing was MINKNOW 2.1 with base-caller, Albacore (v2.0.1).

### Mapping of mRNA Reads

The resulting SAM files were sorted and indexed with “samtools” (v2.1) by sequence coordinate. For visualization on genome browser, they were converted to bigGenePred format (https://genome.ucsc.edu/goldenpath/help/examples/bigGenePred.as) using customized script in TrackCluster package. The coverage track was generated by using “bedtools (2.24)”.

### Defining Novel Isoforms with TrackCluster using the Long Reads

The present invention is to provide the pipeline “TrackCluster” to identify novel isoforms by clustering of isoforms based on their similarity in track structure, i.e., combination of intron/exon and their positions. Briefly, after mapping, each read mapped to the reference genome was converted to a read track in bigGenePred format.

Tracks from each locus were subjected to three rounds of filtering steps to generate novel isoforms (FIG. 2A). First, exons defined by read tracks were compared against all the existing isoforms within a given locus. Any track that did not overlap with any existing isoform for over 50 nt was excluded from being used for novel isoform calling. Second, the following formula were used for calculating pairwise similarity score between two tracks for clustering the tracks into distinct groups. For example, in track A and B, the amount of shared sequence between exons from each track was calculated in nt (A^{exon}∩B^{exon}). The total number of nt were also calculated as the pooled exon sequence in nt between the same two exons (A^{exon}∪B^{exon}). The number of shared and pooled intron sequences were similarly calculated as in exon sequence, and was normalized with a weight of 0.5, assuming average intron length is about twice of that of exon length in C. elegans (WormBase WS260) which was also supported by our long reads (FIG. 20).

If the similarity score was higher than 95% between tracks, the one with a shorter length in summed exons was treated as a subread and merged to the one with a longer length. This step served to cluster the tracks with similar structures into distinct groups.

$$score 1 = \frac{\begin{matrix}( {A}^{e x o n} ⋂ {B}^{e x o n} ) / ( {A}^{e x o n} ⋃ {B}^{e x o n} ) + \\ weight * ( {A}^{i n t r o n} ⋂ {B}^{i n t r o n} ) / ( {A}^{i n t r o n} ⋃ {B}^{i n t r o n} )\end{matrix}}{1 + weight}$$

Third, for the remaining tracks that showed a similarity score lower than 95% (equivalent to the distance score 1 is lower than 5%) between each other, a pairwise similarity score 2 was calculated as follows.

$$score 2 = \frac{\begin{matrix}( {A}^{e x o n} ⋂ {B}^{e x o n} ) / min ( {A}^{e x o n} , {B}^{e x o n} ) + weight * \\ ( {A}^{i n t r o n} ⋂ {B}^{i n t r o n} ) / min ( {A}^{i n t r o n} , {B}^{i n t r o n} )\end{matrix}}{1 + weight}$$

In sequence track A and B, the amount of shared sequence between exons from each track was calculated in nucleotide(nt)(A^{exon}∩B^{exon}) and then the smaller nt number of two summed exon in track A and track B, min(A^{exon}, B^{exon}), was also selected. The number of intron sequences were similarly calculated as in exon sequences and normalized with a weight of 0.5. If the similarity score 2 was higher than 99% (equivalent to the distance score 2 is lower than 1%) between tracks, the one with a shorter length in summed exons was treated as a subread and merged to the one with a longer length. This step served to merge the tracks from partial-length long reads with the one defined by a full-length read. Representative isoform(s) for a locus were/was generated as a result.

TrackCluster was first run using full-length long reads, and then with the remaining reads. There are three purposes for doing this in the present invention. First is to reduce data processing time. Second is to determine the expression level of isoforms using all long reads as described below. Third is to recover the isoforms that are potentially missed by the cutoff. A fraction of isoforms was recovered with a “truncated” 5′ end (“5′ missing” or “UTR truncations” (FIGS. 2A-2F and 3A-3E) relative to all existing transcripts using the remaining reads. For most of these isoforms, they were defined in the presence of an SL, but those newly recovered “truncated” ones were determined without an SL, which were judged by deviations in their remaining part from any existing transcript.

To quantify isoform for each locus, the subreads for each representative isoform was counted. If one subread showed 99% identity to with multiple representative isoforms (number =N), the count for each of these isoforms was counted as 1/N.

### Identification of Spliced Leaders

Previous study showed that Nanopore direct RNA reads was truncated a few nucleotides in the 5′ end, which made the determination of SL problematic. To maximize the possibility to recover an SL, a customized script was written as part of TrackCluster, which used Smith-Waterman (SW) alignment algorithm to detect putative SL signal by aligning the very first 22 nucleotides of the long reads against seven SL sequences. Reads with SW scores over 11 were treated as SL-containing reads. Simulation suggested that FDR was lower than 20% using these parameters and cut-off.

### Identification of PAS Motif

PAS motif was identified as described. A 50 nt region immediately upstream of poly(A) sites were scanned for all possible hexamer sequences to identify the top 50 over-represented motifs. The over-represented motifs were then scanned against the sequences of 14-24 nt (19 +/−5 nt) upstream of a PAS to obtain occurrence of the motifs within these regions. The count of motifs with same composition of nucleotides, for example, AATAAA, AAATAA and ATAAAA, were not merged as described.

### Modification Finding

Modification of the RNA sequences were identified with Tombo package version 1.4. The models of “5mC” and “de novo” were implemented separately to detect possible modification in each read. The score on each site indicated the fraction of a possible modification on a given site. For plotting the modification coverage along gene body and UTRs, the modification coverage was normalized for each isoform using “w0” method with a bin size of 5 nucleotides with EnrichedHeatmap. Only the isoforms with both 5′ and 3′ UTR longer than 50 nucleotides were used in the calculation.

### Characterization of Poly-(A) Tail Length

The poly-(A) lengths of each read were calculated using Nanopolish. The raw current signal from the 3′ unaligned ends of reads were extracted to estimate the length of poly-(A) tail, which was deduced by the duration of the signal.

### Analysis of Differential Expression

An isoform was defined as differentially expressed between stages when the change of its relative abundance (percentage of read count) out of all the transcripts within the same locus is greater than 20% across stage. A gene was defined as differentially expressed between stages when the fold change of its abundance of combined transcripts (read count per million) is greater than four across stage. Only genes supported by at least five long reads were used for the subsequent statistical analyses. Difference in the lengths of long reads between different developmental stages were calculated using Mann—Whitney U test implemented in R 3.4.4.

### Data Access

All raw and processed sequencing data generated in this study have been submitted to the NCBI Gene Expression Omnibus (GEO; https://www.ncbi.nlm.nih.gov/geo/) under accession number GSE130044. The source code for TrackCluster has been deposited in the Github, and is available on https://github.com/Runsheng/trackcluster. All the isoforms and tracks of the long reads can be accessed through the following link (https://genome.ucsc.edu/cgibin/hgTracks?db=cell&hubUrl=https://raw.github.com/runsheng/na notrack/master/hub.txt).

### INDUSTRIAL APPLICABILITY

The present invention provide a method for identifying different isoforms using long reads of RNA sequencing. The potential applications of the present invention include, but are not limited to, detection of fusion transcript or other types of abnormal transcripts as a diagnostic marker for cancer cells/tissues, or genetic diseases. The present method yields a much higher sensitivity as opposed to the existing methods mostly based on Next Generation Sequencing (NGS).

1. A method for identifying different isoforms of RNA from a genome, the method comprising:
providing one or more nucleotide sequence reads from a nucleotide from an organism, wherein said one or more nucleotide sequence reads comprise at least one isoform;
obtaining a reference sequence from an annotated database, wherein the reference sequence includes one or more gene loci having an exon and an intron;
clustering one or more sequence tracks by a distance score Score 1 to obtain a first group of sequence tracks and a second group of sequence tracks;
merging sequence tracks in the first group of sequence tracks if the distance score Score 1 is below a percentage of a first value;
clustering sequence tracks in the second group of sequence tracks by a mutual distance score Score 2;
merging sequence tracks in the second group of sequence tracks if the mutual distance score Score 2 is below a percentage of a second value so as to generate one or more isoforms of the RNA with respect to one or more gene loci.

2. The method of claim 1, wherein said clustering one or more sequence tracks by the distance score Score 1 comprises:
constructing one or more sequence tracks by performing a read-mapping between the one or more nucleotide sequence reads and the one or more gene loci;
excluding the sequence tracks having a first overlap to the exon below a certain amount of nucleotide, or excluding the sequence tracks having a second overlap to the antisense of the one or more gene loci;
computing the distance score Score 1, wherein
the distance score Score 1 is computed by:
$$\frac{\begin{matrix}( {A}^{e x o n} ⋂ {B}^{e x o n} ) / ( {A}^{e x o n} ⋃ {B}^{e x o n} ) + \\ weight * ( {A}^{i n t r o n} ⋂ {B}^{i n t r o n} ) / ( {A}^{i n t r o n} ⋃ {B}^{i n t r o n} )\end{matrix}}{1 + weight} ,$$
wherein A^{exon} is an exon sequence of a first sequence track in the first group of sequence tracks;
B^{exon} is an exon sequence of a second sequence track in the first group of sequence tracks;
(A^{exon}∩B^{exon}) is the amount of shared exon sequence between A^{exon} and B^{exon};
(A^{exon}∪B^{exon}) is the amount of pooled exon sequence between A^{exon} and B^{exon};
A^{intron} is an intron sequence of the first sequence track in the first group of sequence tracks;
B^{intron} is an intron sequence of the second sequence track in the first group of sequence tracks;
(A^{intron}∩B^{intron}) is the amount of shared intron sequence between A^{intron} and B^{intron};
(A^{intron}∪B^{intron}) is the amount of pooled intron sequence between A^{intron} and B^{intron}; and
weight is a ratio of an average intron length to an average exon length of the reference sequence.

3. The method of claim 2, wherein the certain amount of nucleotide is approximately from 10 to 100 nt.

4. The method of claim 2, wherein if the distance score Score 1 is below the percentage of the first value, said merging sequence tracks in the first group of sequence tracks comprises:
subtracting the length of the first sequence track and the length of the second sequence track in the first group of sequence tracks to obtain a third value;
if the third value is over zero, merging the second sequence track with the first sequence track.

5. The method of claim 2, wherein the ratio is approximately from 0.1 to 0.9.

6. The method of claim 4, wherein the distance score Score 1 is below the percentage of approximately 5% of the first value.

7. The method of claim 1, wherein said clustering sequence tracks in the second group of sequence tracks by the mutual distance score Score 2 comprises:
computing the mutual distance score Score 2, wherein
the mutual distance score Score 2 is computed by:
$$\frac{\begin{matrix}( {A}^{e x o n} ⋂ {B}^{e x o n} ) / min ( {A}^{e x o n} , {B}^{e x o n} ) + weight * \\ ( {A}^{i n t r o n} ⋂ {B}^{i n t r o n} ) / min ( {A}^{i n t r o n} , {B}^{i n t r o n} )\end{matrix}}{1 + weight} ,$$
wherein A^{exon} is an exon sequence of a first sequence track in the second group of sequence tracks;
B^{exon} is an exon sequence of a second sequence track in the second group of sequence tracks;
(A^{exon}∩B^{exon}) is the amount of shared exon sequence between A^{exon} and B^{exon};
A^{intron} is an intron sequence of the first sequence track in the second group of sequence tracks;
B^{intron} is an intron sequence of the second sequence track in the second group of sequence tracks;
(A^{intron}∩B^{intron}) is the amount of shared intron sequence between A^{intron} and B^{intron}; and
weight is a ratio of an average intron length to an average exon length of the reference sequence.

8. The method of claim 7, wherein if the mutual distance score Score 2 is below the percentage of the second value, said merging sequence tracks in the second group of sequence tracks comprises:
subtracting the first sequence track and the second sequence track in the second group of sequence tracks to obtain a fourth value;
if the fourth value is over zero, merging the second sequence track with the first sequence track.

9. The method of claim 7, wherein the ratio is approximately from 0.1 to 0.9.

10. The method of claim 8, wherein the mutual distance score Score 2 is below the percentage of approximately 1% of the second value.

11. A method for identifying different RNA isoforms using long reads of RNA sequencing comprising
assigning sequence tracks to a given gene locus based on long-read mapping against a reference genome wherein existing isoforms from the reference genome are also included as a sequence track;
excluding long-read mappings that show few overlaps with existing exons from the existing isoforms or are in antisense to the given gene locus;
clustering the sequence tracks based on a distance score Score 1 wherein the distance score
$$Score 1 = \frac{\begin{matrix}( {A}^{e x o n} ⋂ {B}^{e x o n} ) / ( {A}^{e x o n} ⋃ {B}^{e x o n} ) + \\ weight * ( {A}^{i n t r o n} ⋂ {B}^{i n t r o n} ) / ( {A}^{i n t r o n} ⋃ {B}^{i n t r o n} )\end{matrix}}{1 + weight}$$
wherein the amount of shared sequence in nucleotide between exons or introns from each) sequence track is calculated as (A^{exon}∩B^{exon}) or (A^{intron}∩B^{intron}), and the total number of nucleotide (A^{exon}∪B^{exon}) or (A^{intron}∪B^{intron}) is calculated as pooled sequences in nucleotide between the same two exons or introns, and wherein the weight is based on the ratio of average intron length versus the exon length of the reference genome, with a default value of 0.5;
merging the sequence tracks with a cut-off based on the distance scores Score 1 between the sequence tracks, wherein the sequence track with a first length in summed exons of the isoforms other than the referenced isoforms is treated as a first subread and merged with the sequence track with a second length in the summed exons of said isoforms; wherein said second length is longer than the first length;
merging the sequence tracks if the distance score Score 1 is lower than 5% and retaining one of the other isoforms with the biggest size of the summed exons from each group along with the existing isoforms for subsequent isoform calling, wherein the remaining sequence tracks are assigned as a second subread to be used for expression quantification and intron/exon boundary correction,
clustering the retained sequence tracks based on a mutual distance score Score 2 wherein the mutual distance score
$$Score 2 = \frac{\begin{matrix}( {A}^{e x o n} ⋂ {B}^{e x o n} ) / min ( {A}^{e x o n} , {B}^{e x o n} ) + weight * \\ ( {A}^{i n t r o n} ⋂ {B}^{i n t r o n} ) / min ( {A}^{i n t r o n} , {B}^{i n t r o n} )\end{matrix}}{1 + weight} ,$$
merging the sequence track with a first length in the summed exons of the isoforms obtained after said clustering, being assigned as a third subreads, into a sequence track with a second length in the summed exons of said isoforms if the mutual distance score Score 2 is lower than 1% so as to generate one or more resulting isoforms for the given gene locus;
wherein said second length is longer than the first length;
correcting the resulting sequence tracks for intron/exon junctions to result in different RNA isoforms.

12. The method according to claim 11 wherein for said intron/exon boundary correction, the junctions from the second subreads are aligned against the junctions defined for the isoform by the longest full-length long-read.

13. The method according to claim 11 wherein a minor shift of no more than 5% change in distance score in exon-intron boundary caused by read errors are permitted to reduce over calling of different RNA isoforms.
